(19) United States
(12) Patent Application Publication
(10) Pub. No.: US 2007/0065520 A1
(43) Pub. Date: Mar. 22, 2007

(54) Mri contrast medium composition for oral administration

(76) Inventor: Nils-Olof Johansson, Lidingo (SE)

Correspondence Address: BUCHANAN, INGERSOLL & ROONEY PC, POST OFFICE BOX 1404, ALEXANDRIA, VA, 22313-1404, US

(21) Appl. No.: 10/583,317

(22) PCT Filed: Nov. 10, 2004
(86) PCT No.: PCT/SE04/01644

§ 371 Date: Jun. 15, 2006

(30) Foreign Application Priority Data
Dec. 19, 2003 (SE) 0303429-5

(51) Int. Cl. A61K 33/32

(52) U.S. Cl. 424/639

(57) ABSTRACT

The use of a physiologically acceptable manganese (II) compound and an uptake promoter in the form of one or more amino acids for the manufacture of an MRI contrast composition for oral administration and MRI examination of the liver in a ratio of Mn to promoter higher than that at which coordination compounds between Mn and promoter are formed to substantial degree is described; and an MRI contrast medium composition for such use; MRI contrast medium kit; and a method for imaging of a mammalian liver using such contrast medium composition are provided.

### FIELD OF THE INVENTION

The present invention relates to magnetic resonance imaging (MRI), more specifically to the oral administration of manganese contrast medium compositions for imaging of the liver and a method for MRI of a liver using the manganese contrast medium composition.

### BACKGROUND OF THE INVENTION

Magnetic resonance imaging (MRI) is now well established as a medical diagnostic tool. The ability of the technique to generate high quality images and to differentiate between soft tissues without requiring the patient to be exposed to ionizing radiation has contributed to this success.

Manganese is a well-known paramagnetic contrast medium useful for MRI of soft tissues in the body. When administered intravenously as a contrast medium, manganese may however be teratogenic at clinical dosages. Administered intravenously, manganese is also known to interfere with the normal functioning of the heart by replacement of calcium in the muscular cell of the heart. To avoid this problem Mn bound in chelate complexes has been used; however the chelation prevents or reduces the enhancement of Mn binding with tissues.

EP 0 308 983 A2 thus proposes a contrast medium for parenteral application for imaging of the heart and liver in the form of a non-chelate coordination compound to reduce toxicity. Here Mn is bound in a complex, i.e. a coordination complex, to avoid the toxic effect of free Mn. The proposed compound has the formula Mn(II) (H₂O)ₘAlₙA2ₒA3ₚA4_{q})^{-a}(Y, Zᵣ)^{-a}, wherein A1, A2, A3 and A4 are the same or different amino-substituted carboxylic acid groups having from 2 to 18 carbons; Y and Z are each the same or a different anion of a pharmaceutically acceptable inorganic acid or an organic carboxylic acid having from 2 to 18 carbons; a is the valence of the ions; m, n, o, p and q are each 1 or 0, (m+n+o+p+q)=4; and r is 1, and if Y is a multivalent anion, r is 0 or 1.

EP 0 308 983 A2 discloses organ specifically designed compositions for administration by injection and selected to carry the coordination compound to the portion of the body to be imaged by the MR device.

In order to further reduce the risk of dangerous or even fatal effect on the heart, oral administration of manganese has previously been proposed. A result of the vascularisation of the upper gastrointestinal tract is that orally administered material taken up into the blood from the gut is adsorbed in the liver before passing to the heart. In the case of manganese, absorption by the hepatocytes in the liver prevents cardiotoxic levels of manganese reaching the heart. This hepatocyte uptake of manganese has led to the proposed use of orally administered manganese as a liver imaging contrast medium.

WO 96/05867 discloses a contrast medium composition comprising a physiologically tolerable manganese compound, an uptake promoter and a physiologically tolerable carrier or excipient, having a manganese concentration of at least 0.3 mM or being in a dosage unit form containing at least 300 μmol manganese, wherein the uptake promoter comprises a physiologically tolerable reducing compound containing an α-hydroxy ketone group, a physiologically tolerable acid containing α- and/or β-hydroxy or amino groups, or a salt thereof, and/or vitamin D. The disclosure clearly indicates that improved uptake of a manganese will be obtained by using an uptake promoter in molar excess over manganese.

WO 97/02842 discloses the use of a Mn-contrast medium containing a promoter for oral administration for imaging of the stomach, liver, bile duct and gall bladder. The promoter is in the form of at least one amino acid and/or a vitamin D. However, nor does this document teach any advantage of avoiding substantial formation of coordination compounds between manganese and uptake promoter.

### SUMMARY OF THE INVENTION

It has now surprisingly been found that by increasing previously taught molar ratios of Mn to amino acid it is possible to improve the liver uptake of manganese, and such improvement is independent of whether vitamin D₃ is present or not.

In this connection it is important to note that even a relatively small increase in the amount of manganese present in the liver tissue will result in a significant enhancement of the image.

In one aspect the present invention provides for the use of a physiologically acceptable manganese (II) compound and an uptake promoter in the form of one or more amino acids for the manufacture of an MRI contrast composition for oral administration and MRI examination of the liver, in a ratio of Mn/promoter higher than that at which coordination compounds between Mn and promoter are formed to a substantial degree.

In a second aspect the invention provides for an MRI contrast medium composition for oral administration for examination of the liver, comprising as an active ingredient a physiologically acceptable manganese (II) compound and an uptake promoter comprising one or more amino acids, wherein Mn and the promoter are used in a molar ratio higher than that at which coordination compounds between Mn and promoter are formed to a substantial degree.

In a third aspect the invention provides for an MRI contrast medium kit comprising a first container accommodating a physiologically acceptable manganese (II) compound, a second container accommodating an uptake promoter comprising one or more amino acids, and, optionally, instructions for use of the kit, the molar ratio of Mn/promoter being within the range of about 2:3 to about 3:1.

In a fourth aspect the invention provides for a method for MRI of a mammalian liver using a contrast medium composition as defined above, said method comprising oral administration of an effective amount of a contrast composition as described above to a mammal, including man, in need of such an MRI.

### DETAILED DESCRIPTION OF THE INVENTION

The prior art describes on the one hand the necessity of providing manganese in the form of coordination complexes to avoid toxic effects by injection and, on the other hand, the advantage of using the uptake promoter in a molar excess over the manganese when using oral administration.

The main object of the present invention is to provide contrast medium compositions for oral administration of manganese for improved imaging of the liver due to increased absorption from the gastrointestinal tract.

The present invention is taking advantage of the novel finding that ratios of manganese/uptake promoter which form coordination complexes are not very effective in regard to adsorption in the liver. Accordingly, another object of the present invention is to provide compositions, wherein manganese and uptake promoter are present in such portions as not to form coordination complexes to any significant extent. Expressed in another way the ratio between manganese and promoter shall be higher than that at which coordination compounds between manganese and promoter are formed to a substantial degree. In this context “substantial” is to be interpreted preferably as major, i.e. leaving more than 50% of the manganese ions non-coordinated and available for absorption.

Thus, the present invention is based on the surprising finding that coordination compounds involving manganese and uptake promoter are to be avoided in order that improved adsorption of manganese in the liver will be obtained.

In the composition according to the invention, the preferred molar ratio of manganese to uptake promoter is higher than or equal to about 2:3; more preferably higher than or equal to about 1:1, most preferably higher than or equal to about 2:1. The upper limit is preferably at most about 3:1. Thus, a preferable range is from about 2:3 to about 3:1.

The preferred dosage of the composition according to the present invention will vary according to a number of factors, such as the age, weight and species of the subject, and the particular uptake promoter used. Conveniently, the dosage of manganese will normally be in the range of from about 25 to about 150 μmol/kg body weight. Preferably the dosage of manganese will be in a range of from about 50 to about 125 μmol/kg body weight, and more preferably the dosage of manganese will be in the range of from about 50 to about 100 μmol/kg body weight.

The contrast medium composition according to the invention may comprise a manganese compound together with a mixture of two or more uptake promoters, e.g. a mixture of several amino acids.

Compounds which have been found to be suitable for use as uptake promoters include all physiologically acceptable amino acids. Amino acids which are effective as uptake promoters in the compositions of the invention include all the native amino acids, i.e. alanine, valine, leucine, tryptophan, methionine, isoleucine, proline, phenylalanine, serine, glycine, threonine, cysteine, asparagine, glutamine, tyrosine, aspartic, acid, glutamic acid, arginine, lycine and histidine.

A preferred group of amino acids for use as uptake promoters in the compositions of this invention is selected from neutral amino acids including asparagine, and aspartic acid. Particularly preferred amino acids are asparagine, aspartic acid and alanine, especially L-alanine.

The invention is preferably practiced while using the amino acid(s) as the only promoter(s), but this does not exclude the use of said amino acid(s) together with any other common promoter(s), e.g. vitamin D₃.

Alanine has been used as an example in the present invention. WO 96/05867 shows a general effect of other amino acids, such as glycine, valine, glutamine, aspartic acid, glutamic acid, lysine, arginine, cysteine and methionine. Decisive for the choice of amino acid or amino acids are the price and the stability as well as the taste, appearance and odor of the amino acid to have a product acceptable to the patients.

Paramagnetic materials, such as manganese ions, may act as either positive or negative MRI contrast agents depending upon a number of factors, including the concentration of the ions at the imaging site and the magnetic field strength used in the imaging procedure. At the concentrations of manganese contemplated for use in the compositions of the invention, the manganese-containing contrast medium will in general function as a positive contrast medium.

The compositions of the invention are particularly suited to use as a dispersion in an aqueous medium. For such a purpose the composition may be administered into the gastrointestinal tract orally or via a gastric tube.

It is possible to formulate the contrast medium immediately or shortly prior to administration by mixing the uptake promoter with the manganese compound.

As the contrast medium is to be administered orally a patient can administer it himself. The patient is therefore not obliged to stay in the hospital for several hours before being scanned. He can take the oral medium himself without need for medical assistance.

The contrast medium composition of the invention may include components other than the uptake promoter, and the manganese compound, for example conventional pharmaceutical formulation aids, such as wetting agents, buffers, disintegrants, binders, fillers, flavoring agents and liquid carrier media, such as sterile water, water/ethanol etc.

The pH of the composition is preferably in the acidic range, e.g. 2 to7, and while the uptake promoter may itself serve to yield a composition with this pH, buffers or pH adjusting agents may be used.

The contrast media may be formulated in any edible/drinkable medium and in conventional pharmaceutically administrable forms, such as tablets, capsules, powders, solutions, dispersions, syrups, etc. Since the contrast medium is to be administered orally, a patient can administer the contrast medium himself prior to scanning.

The manganese compound, which for oral administration is preferably dissolved or suspended in water, may for example be in the form of a salt, or may be a mixture of different salts. Particularly preferred are salts in which the manganese is present as Mn(II) rather than Mn(III) since the former has a better adsorption profile and is thus more efficient as an MR contrast medium for the liver.

Examples of manganese compounds preferred for use in accordance with the invention include pharmaceutically acceptable salts, e.g. manganese chloride, manganese ascorbate, manganese kojate, manganese salicylate and manganese gluconate, especially the chloride.

The invention will now be further described by examples which illustrate the effectiveness of the uptake of manganese for various concentrations of uptake promoter.

### EXAMPLE 1

The study was conducted at Scantox, Hestehavevej 6A, DK-4623 Lille Skensved, Denmark.

The objective of the studies was to evaluate the uptake of manganese chloride in rats by measurement of the manganese content in the liver as an comparison example.

The experiment was performed in 55 outbred Sprague Dawley rats from Taconic M&B A/S, Denmark. The animals in the study were allocated to 11 groups. The test composition was administered once orally by gavage. Animals in Group 1 (control) were only treated with sterile water. Group 2 received only 100 μmol MnCl₂/kg. The other nine treated groups received concurrent oral doses of 100 μmol MnCl₂/kg (Group 3 to 11) and/or 450, 300 and 150 μmol alanine/kg and/or 20, 40 and 60 IU vitamin D₃/kg in different permutations (table 1).

Three hours after dose administration, the animals-were killed and the livers were weighed and retained frozen. The samples were subsequently homogenised and analysed for manganese concentrations.

The highest group mean amount of manganese was seen in group5, receiving respectively 150 μmol/kg alanine and 60 IU vitamin D₃/kg, indicating that a low amount of alanine, i.e. 150 μmol/kg is increasing the manganese uptake in the liver, compared to groups 3 and 4, receiving 450 and 300 μmol/kg alanine, respectively and the same amount of vitamin D₃ (see table 1). The results in table 1 show clearly that there is a significant increase in manganese uptake from the gut with decreasing amounts of alanine. For the three subgroups 60, 40 and 20 IU vitamin D₃/kg bodyweight, the best manganese uptake is seen with the lowest alanine amount, i.e. 150 μmol/kg bodyweight (group 5 compared to 3 and 4, group 8 compared to group 6 and 7, group 11 compared to group 9 and 10). However a vitamin D₃ content of 40 IU/kg bodyweight shows no difference between group 7 and 8, which suggest that this level of vitamin D₃ there is now significant difference between300 and 150 μmol/kg alanine.

TABLE 1
(grouped according to vit. D₃ content)

| | Treatment | | | | |
|---|---|---|---|---|---|
| | MnCl₂₋ | Alanine | Vitamin D₃ | Mol. | Liver |
| | (μmol/kg | (μmol/kg | (IU/kg | Ratio | Mean Mn |
| Group | b.w.) | b.w.) | b.w.) | Mn:Ala | nmol/g |
| 1 | 0 | 0 | 0 | — | 41.4 |
| 2 | 100 | 0 | 0 | — | 44.4 |
| 3 | 100 | 450 | 60 | 1:4.5 | 61.1 |
| 4 | 100 | 300 | 60 | 1:3 | 61.8 |
| 5 | 100 | 150 | 60 | 1:1.5 | 76.1 |
| 6 | 100 | 450 | 40 | 1:4.5 | 66.4 |
| 7 | 100 | 300 | 40 | 1:3 | 73.8 |
| 8 | 100 | 150 | 40 | 1:1.5 | 73.7 |
| 9 | 100 | 450 | 20 | 1:4.5 | 68.4 |
| 10 | 100 | 300 | 20 | 1:3 | 69.8 |
| 11 | 100 | 150 | 20 | 1:1.5 | 72.6 |

Taken together, table 1 shows that when the ratio of Mn:ala is 1:4.5, the Mn liver uptake is enhanced by addition of vit D₃ (group 3, 6 and 9). However when the ratio of Mn:ala is 1:1.5 addition of vit.D₃ in a high amount (i.e. 60 or 40 IU/kg b.w.) is not needed in order to obtain fairly good images (group 5, 8 and 11).

### EXAMPLE 2

The study was conducted as described in example 1. However the experiment was performed in55 outbred Sprague Dawley rats from Taconic M&B A/S, Denmark. The animals in the study were allocated to 11 groups. The test composition was administered orally by gavage. Animals in Group 1 (control) were only treated with sterile water. Group 2 received only 100 μmol MnCl₂/kg. The other nine treated groups received concurrent oral doses of 100 μmol MnCl₂/kg (Group 3 to 11), 150, 75 and 37.5 μmol alanine/kg and 20, 10 and 5 IU vitamin D₃/kg in different permutations (table 2A).

Three hours after dose administration, the animals were killed and the livers were weighed and retained frozen. The samples were subsequently homogenised and analysed for manganese concentrations.

Manganese concentrations in the liver were higher in groups 3 to 11 compared to Groups 1 and 2. This correlates well with the fact that Groups 1 and 2 received either 0 or 1050 μmol MnCl₂/kg without addition of uptake promoters. The highest group mean amount of manganese was seen in group 6 to 11, receiving respectively 75 and 37.5 μmol/kg alanine, indicating that a high amount of alanine, i.e. 150 μmol/kg is not increasing the manganese uptake in the liver (group 3 to 5) compared to the alanine amounts provided to group 6 to 11 (see table 2A (or 2B, NB: different grouping)). The results clearly show that there is a significant increase in manganese uptake from the gut with the use of promoters wherein the molecular ratio between Mn and amino acid, for example alanine is higher than1:1.

TABLE 2A
(Grouped according to alanine content)

| | Treatment | | | | |
|---|---|---|---|---|---|
| | MnCl₂ | Alanine | Vitamin D₃ | Mol. | Liver |
| | (μmol/kg | (μmol/kg | (IU/kg | Ratio | Mean Mn |
| Group | b.w.) | b.w.) | b.w.) | Mn:Ala | nmol/g |
| 1 | 0 | 0 | 0 | — | 34.6 |
| 2 | 100 | 0 | 0 | — | 63.4 |
| 3 | 100 | 150 | 20 | 1:1.5 | 69.9 |
| 4 | 100 | 150 | 10 | 1:1.5 | 72.7 |
| 5 | 100 | 150 | 5 | 1:1.5 | 68.4 |
| 6 | 100 | 75 | 20 | 1:0.75 | 69.2 |
| 7 | 100 | 75 | 10 | 1:0.75 | 73.8 |
| 8 | 100 | 75 | 5 | 1:0.75 | 72.0 |
| 9 | 100 | 37.5 | 20 | 1:0.375 | 72.6 |
| 10 | 100 | 37.5 | 10 | 1:0.375 | 79.8 |
| 11 | 100 | 37.5 | 5 | 1:0.375 | 73.1 |

TABLE 2B
(grouped according to Vit. D₃ content)

| | Treatment | | | | |
|---|---|---|---|---|---|
| | MnCl₂ | Alanine | Vitamin D₃ | Mol. | Liver |
| | (μmol/kg | (μmol/kg | (IU/kg | Ratio | Mean Mn |
| Group | b.w.) | b.w.) | b.w.) | Mn:Ala | nmol/g |
| 1 | 0 | 0 | 0 | — | 34.6 |
| 2 | 100 | 0 | 0 | — | 63.4 |
| 3 | 100 | 150 | 20 | 1:1.5 | 69.9 |
| 6 | 100 | 75 | 20 | 1:0.75 | 69.2 |
| 9 | 100 | 37.5 | 20 | 1:0.375 | 72.6 |
| 4 | 100 | 150 | 10 | 1:1.5 | 72.7 |
| 7 | 100 | 75 | 10 | 1:0.75 | 73.8 |
| 10 | 100 | 37.5 | 10 | 1:0.375 | 79.8 |
| 5 | 100 | 150 | 5 | 1:1.5 | 68.4 |
| 8 | 100 | 75 | 5 | 1:0.75 | 72.0 |
| 11 | 100 | 37.5 | 5 | 1:0.375 | 73.1 |

### EXAMPLE 3

The study was conducted as described in Ex. 1 and 2. However the experiment was performed in 40 male outbred Sprague Dawley rats from Taconic M&B A/S, Denmark. The animals in the study were allocated to 8 groups. The test article was administered once orally by gavage. Animals in Group 1 (control) were only treated with sterile water. Group 2 received only 100 μmol MnCl₂/kg b.w. The other six treated groups received concurrent oral doses of 50 μmol (Group 3) or 100 μmol MnCl₂/kg (Group 4 to 8), 25, 50, 200 and 200 μmol Alanine/kg and 0 or 10 IU vitamin D₃/kg in different permutations (see also table 3).

Manganese concentrations in the liver were higher in groups 2, 4, 5, 6, 7 and 8 compared to Groups 1 and 3. This correlates well with the fact that Groups 1 and 3 received the lowest amount of manganese i.e. 0 or 50 μmol MnCl₂/kg, respectively. The highest group mean amount of manganese was seen in group 5 and 7 (Table 3).

It is worth noting that group 3 and 4 showed a remarkable difference in manganese uptake, 95.3 and 110.3 respectively, even though that the molecular ratio between alanine and manganese was the same in the 2 groups, i.e. 2:1. This shows that the amount of manganese in the solution is important for an improved manganese uptake (Table 3). 100 μmol/kg bodyweight is far better than50 μmol/kg bodyweight.

Furthermore, the manganese uptake indicates that alanine also has to be provided in a certain amount, i.e. >25 μmol/kg b.w. since the manganese uptake in group 5 giving 50 μmol/kg alanine is better than in group 6 only provided 25 μmol/kg, i.e 128.8 nmol/g and 115.9 nmol/g respectively (Table 3).

In conclusion, Group 5 and 7 (treated with a combination of 100 μmol MnCl₂/kg and 50 μmol alanine/kg and, respectively 10 IU vitamin D₃) was the groups having the highest amount of manganese uptake in the liver. However, also compositions without vitamin D₃ show good results.

TABLE 3

| | Treatment | | | | |
|---|---|---|---|---|---|
| | MnCl₂ | Alanine | Vitamin D₃ | Mol. | Liver |
| | (μmol/kg | (μmol/kg | (IU/kg | Ratio | Mean Mn |
| Group | b.w.) | b.w.) | b.w.) | Mn:Ala | nmol/g |
| 1 | 0 | 0 | 0 | — | 45.0 |
| 2 | 100 | 0 | 0 | — | 114.0 |
| 3 | 50 | 100 | 0 | 1:2 | 95.3 |
| 4 | 100 | 200 | 0 | 1:2 | 110.3 |
| 5 | 100 | 50 | 0 | 1:0.5 | 128.8 |
| 6 | 100 | 25 | 0 | 1:0.25 | 115.9 |
| *7 | 100 | 50 | 10 | 1:0.5 | 133.8 |
| 8 | 100 | 25 | 10 | 1:0.25 | 117.8 |

*Group 7: 1 animal excluded since no Mn was taken up

Discussion:

The above specific examples clearly indicate the advantage of avoiding substantial formation of coordination compounds between manganese and uptake promoter in order to obtain improved gastro-intestinal uptake of manganese for administration to the liver following oral administration of the contrast medium composition. This finding is contrary to what could be expected in a consideration of the state of the art.

1. (canceled)

2. The method according to claim 1, wherein said ratio is in the range of from 1:1 to 3:1.

3. The method according to claim 2, wherein said ratio is in the range of from 2:1 to 3:1.

4. The method according to claim 21, wherein the dosage of manganese is in the range of from 25 to 150 μmol/kg body weight.

5. The method according to claim 4, wherein the dosage of manganese is in the range of from 50 to 125 μmol/kg body weight.

6. The method according to claim 5, wherein the dosage of manganese is in the range of from 50 to 100 μmol/kg body weight.

7. The method according to claim 21, wherein the uptake promoter is selected from the group consisting of alanine, valine, leucine, tryptophan, methionine, isoleucine, proline, phenylalanine, serine, glycine, threonine, cysteine, asparagine, glutamine, tyrosine, aspartic acid, glutamic acid, arginine, lycine and histidine.

8. The method according to claim 7, wherein said uptake promoter is a neutral amino acid.

9. The method according to claim 8, wherein said promoter is L-alanine.

10. An MRI contrast medium composition for oral administration for examination of the liver comprising as an active ingredient a physiologically acceptable manganese (II) compound and an uptake promoter comprising one or more amino acids wherein Mn and the promoter are used in a molar ratio higher than that at which coordination compounds between Mn and promoter are formed to a substantial degree, wherein the molar ratio of Mn to promoter is in the range of from 2:3 to 3:1.

11. A composition according to claim 10, wherein said ratio is in the range of from 1:1 to 3:1.

12. A composition according to claim 11, wherein said ratio is in the range of from 2:1 to 3:1.

13. A composition according to claim 10, wherein the dosage of manganese is in the range of from 25 to 150 μmol/kg body weight.

14. A composition according to claim 13, wherein the dosage of manganese is in the range of from 50 to 125 μmol/kg body weight.

15. A composition according to claim 14, wherein the dosage of manganese is in the range of from 50 to 100 μmol/kg body weight.

16. A composition according to claim 10, wherein the uptake promoter is selected from the group consisting of alanine, valine, leucine, tryptophan, methionine, isoleucine, proline, phenylalanine, serine, glycine, threonine, cysteine, asparagine, glutamine, tyrosine, aspartic acid, glutamic acid, arginine, lycine and histidine.

17. A composition according to claim 16, wherein said promoter is selected from neutral amino acids including asparagine and aspartic acid.

18. A composition according to claim 17, wherein said promoter is L-alanine.

19. An MRI contrast medium kit comprising a first container accommodating a physiologically acceptable manganese (II) compound, and a second container accommodating an uptake promoter comprising one or more amino acids, and optionally, instructions for the use of the kit, the molar ratio of Mn to promoter being within the range of 2:3 to 3:1.

20. A kit according to claim 19, wherein said molar ratio, the range of from 2:1 to 3:1.

21. A method for MRI of a mammalian liver comprising orally administering an effective amount of said MRI contrast medium composition according to claim 10.

22. The method according to claim 8, wherein said neutral amino acid is selected from the group consisting of asparagine and aspartic acid.
